(19) United States
(12) Patent Application Publication
(10) Pub. No.: US 2022/0296825 A1
(43) Pub. Date: Sep. 22, 2022

(54) EXHALATION DELIVERY SYSTEM FOR AND METHOD OF TREATING SINUS DISEASE

(71) Applicant: OptiNose AS, Oslo (NO)

(72) Inventors: Per Gisle DJUPESLAND, Oslo (NO); John MESSINA, Downingtown, PA (US)

(21) Appl. No.: 17/281,290

(22) PCT Filed: Oct. 4, 2019
(86) PCT No.: PCT/EP2019/076980

§ 371 Date: Mar. 30, 2021

Related U.S. Application Data
(60) Provisional application No. 62/741,370, filed on Oct. 4, 2018.

(51) Int. Cl. A61M 15/00; A61M 15/08

(52) U.S. Cl. CPC A61M 15/009; A61M 15/0021; A61M 15/08; A61M 15/0098; A61M 2202/064; A61M 2016/0027

(57) ABSTRACT

An exhalation delivery system for and method of treating a patient having sinus disease, comprising, following Draf 2 or Draf 3 surgery, administering a therapeutic agent including a corticosteroid to the patient, wherein the therapeutic agent is administered by an exhalation delivery device comprising a mouthpiece and a nosepiece, fitting the nosepiece to a first nostril of the patient, and the patient exhaling into the mouthpiece to create a fluid flow out of the nosepiece and into the first nostril during administration of the therapeutic agent to the patient.

The present invention relates to exhalation delivery system for and method of treating a patient by a combined surgical and post-surgical treatment of sinus disease, and in particular chronic rhinosinusitis (CRS).

CRS is a chronic inflammatory condition of the nasal cavity and paranasal sinuses. Corticosteroids are a mainstay of medical therapy in the management of CRS, because the broad anti-inflammatory activity addresses the continual production of inflammatory mediators and polyp formation in the nasal passages.¹ For safety reasons, topically-acting corticosteroids are preferred over oral corticosteroids for long-term management, and it is thought to be important to deliver the medication into the affected paranasal sinus cavities in order to ensure effective long-term management of CRS.¹ However, owing to the complex and convoluted nasal anatomy, unoperated sinuses are difficult to access with medication delivered with high-volume nasal rinses or nasal sprays compared to sinuses that have been surgically opened.²

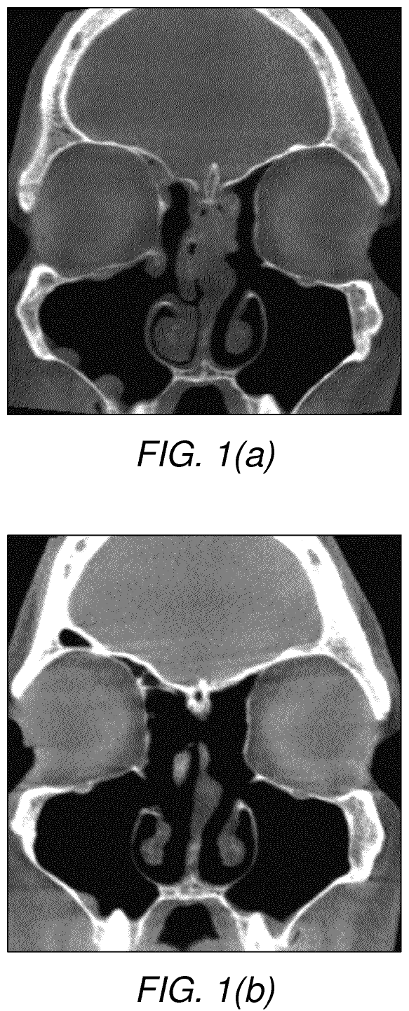

Endoscopic sinus surgery (ESS) is the standard for surgical treatment of CRS and is generally indicated when symptoms persist despite appropriate medical therapy.² Surgery aims to establish a patent nasal airway and relieve sinus outflow obstruction, decrease the overall inflammatory load, and open the sinuses in order to allow for removal of debris/bacteria via irrigation and improved postoperative topical drug delivery.² Surgical approaches can range from dilation of the natural ostia to interventions that completely remodel the anatomy and drainage pathways, particularly aimed at improving access to the paranasal sinuses for delivery of topically-acting drugs and for mechanical irrigation.¹ In addition to endoscopic maxillary antrostomies and ethmoidectomy, endoscopic frontal sinusotomy, including Draf 2 and Draf 3, as illustrated in FIG. 1, is indicated for patients with refractory forms of chronic frontal sinus disease. The Draf 2 procedure involves resection of the floor of the frontal sinus from the nasal septum medially, to the lamina papyracea laterally. The dissection involves removal of the anterior face of the frontal recess. Thus, the frontal sinus ostium is enlarged to its maximum dimension. The Draf 3 procedure extends the Draf 2 procedure by the additional resection of the superior nasal septum and entire frontal sinus floor.

The extent to which a liquid introduced by nasal delivery accesses the sinus cavities after surgery is dependent on a number of factors, including the head position, drug delivery device, and the degree of surgery.² High-volume, low-flow (HVLF) nasal irrigation, for example, using a squeeze bottle, may be more effective for distributing liquid-containing medication into the sinuses compared to low-volume delivery methods, such as nasal sprays, nebulizers or drops.^{2,3} There is prior evidence that saline irrigation is beneficial in treating the symptoms of CRS when used as the sole modality of treatment.⁴ On this basis, it has been advocated to add high-dose, topically-acting corticosteroids, mainly budesonide, to the HVLF saline nasal irrigation in order to combine irrigation and drug delivery in the treatment of post-surgical CRS.

Furthermore, computational fluid dynamics (CFD) simulations of sinus deposition of HVLF-delivered liquid suggest that frontal sinus deposition can be increased after the Draf 3 procedure, insofar as the surgical opening allows for open fluid communication to the frontal sinus region.

The present inventors have recognized that low-volume deposition using an exhalation delivery system (EDS) device, such as disclosed in WO-A-2013/124491, as available from OptiNose US (Yardley, PA) as XHANCE™, or as disclosed in WO-A-2006/030210, as available from Avanir Pharmaceuticals (Aliso Viejo, Calif.) as ONZETRA™ XSAIL™, surprisingly provides for improved maxillary and ethmoid sinus penetration, and hence improved treatment efficacy as compared to high-volume deposition, which is contrary to that which would be expected by the skilled person.

In one aspect the present invention provides an exhalation delivery system for or method of treating a patient having sinus disease, comprising, following Draf 2 or Draf 3 surgery, administering a therapeutic agent including a corticosteroid to the patient, wherein the therapeutic agent is administered by an exhalation delivery device comprising a mouthpiece and a nosepiece, fitting the nosepiece to a first nostril of the patient, and the patient exhaling into the mouthpiece to create a fluid flow out of the nosepiece and into the first nostril during administration of the therapeutic agent to the patient.

In one embodiment the corticosteroid is fluticasone.

In one embodiment the corticosteroid is fluticasone propionate.

In one embodiment the surgery is Draf 2 surgery.

In another embodiment the surgery is Draf 3 surgery.

In one embodiment the therapeutic agent is in the form of a liquid aerosol.

In one embodiment the therapeutic agent is administered using a spray pump.

In another embodiment the therapeutic agent is in the form of a powder.

In one embodiment the therapeutic agent is delivered in an amount of up to 400 μg, optionally twice daily.

In another embodiment the therapeutic agent is delivered in an amount of up to 200 μg, optionally twice daily.

In a further embodiment the therapeutic agent is delivered in an amount of up to 100 μg, optionally twice daily.

In one embodiment the therapeutic agent is delivered in an amount of at least 50 μg, optionally twice daily.

In one embodiment the method further comprises controlling a duration, rate and/or pressure of the fluid flow.

In one embodiment the method comprises controlling the fluid flow duration to be in a range of from about 2 seconds to about 3 seconds.

In one embodiment the method further comprises controlling the fluid flow rate at at least 10 Lmin⁻¹, optionally at least 20 Lmin⁻¹, and optionally at least 30 Lmin⁻¹.

In one embodiment the method further comprises fitting the nosepiece to a second nostril of the patient, and the patient exhaling into the mouthpiece to create a fluid flow out of the nosepiece and into the second nostril during administration of the therapeutic agent to the patient.

In one embodiment the first and second nostrils are different nostrils of the patient or the same nostril of the patient.

In one embodiment the method further comprises performing Draf 2 or Draf 3 surgery to the patient prior to administration of the therapeutic agent to the patient.

In one embodiment the surgery is endoscopic sinus surgery.

In one embodiment the sinus disease is chronic rhinosinusitis (CRS).

Preferred embodiments of the present invention will now be described hereinbelow by way of example only with reference to the accompanying drawings, in which:

FIGS. 1(a) and (b) illustrate coronal computed tomography (CT) scans following Draf 2 and Draf 3 surgical procedures, respectively;

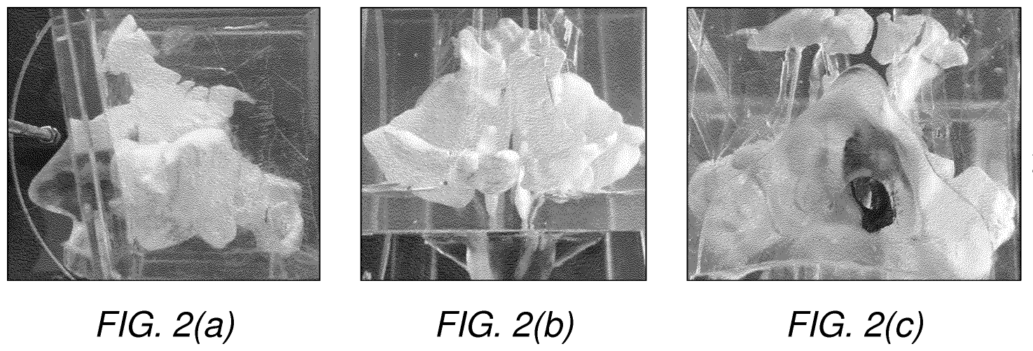

FIGS. 2(a) to (c) illustrate left lateral, superior and frontal views, respectively, following delivery using a nasal spray (Nasonex™, 0.1 mL×2) into the left nostril in a Draf 2 cast;

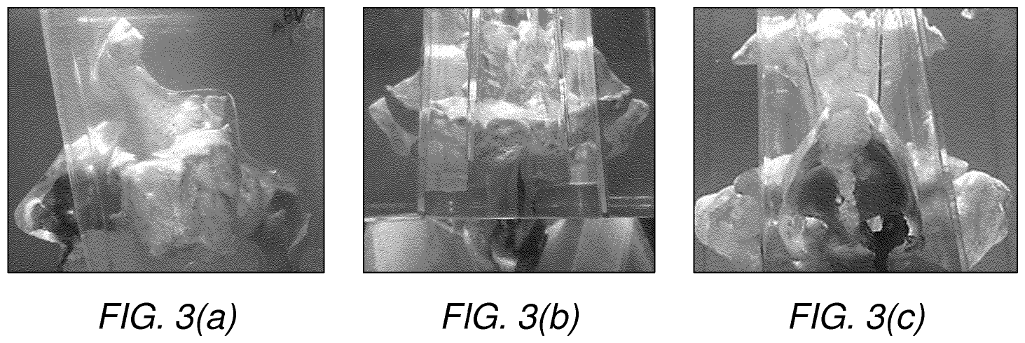

FIGS. 3(a) to (c) illustrate left lateral, superior and frontal views, respectively, following delivery using a nasal spray (Nasonex™, 0.1 mL×2) into the left nostril in a Draf 3 cast;

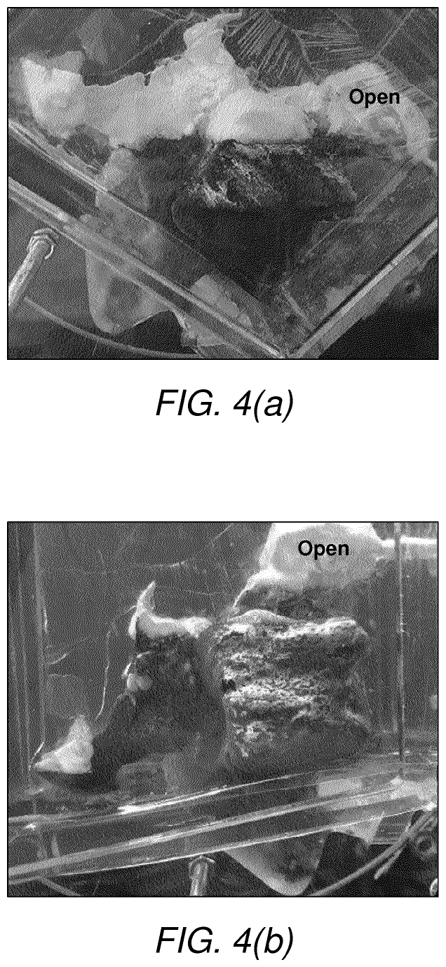

FIGS. 4(a) and (b) illustrate left lateral views with 45 and 90 degree head (vertex) positions, respectively, following high-volume, low-flow (HVLF) delivery into the left nostril in a Draf 2 cast;

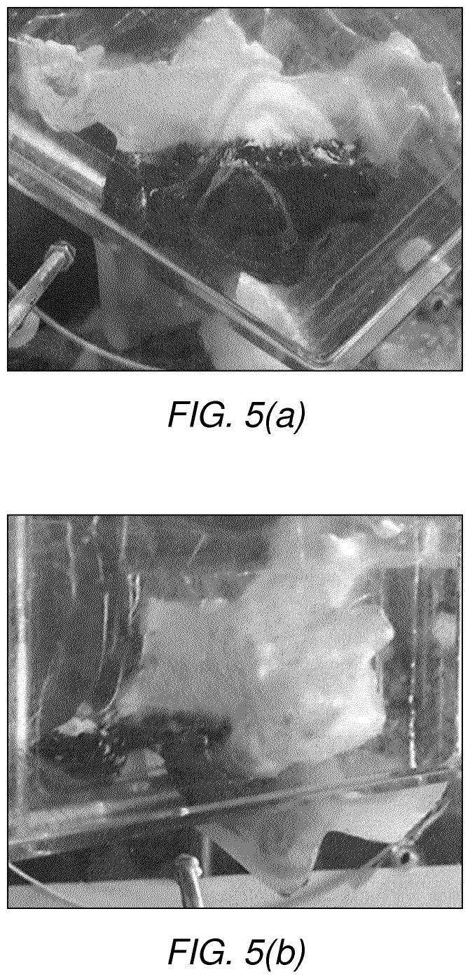

FIGS. 5(a) and (b) illustrate left lateral views with 45 and 90 degree head (vertex) positions, respectively, following high-volume, low-flow (HVLF) delivery into the left nostril in a Draf 3 cast;

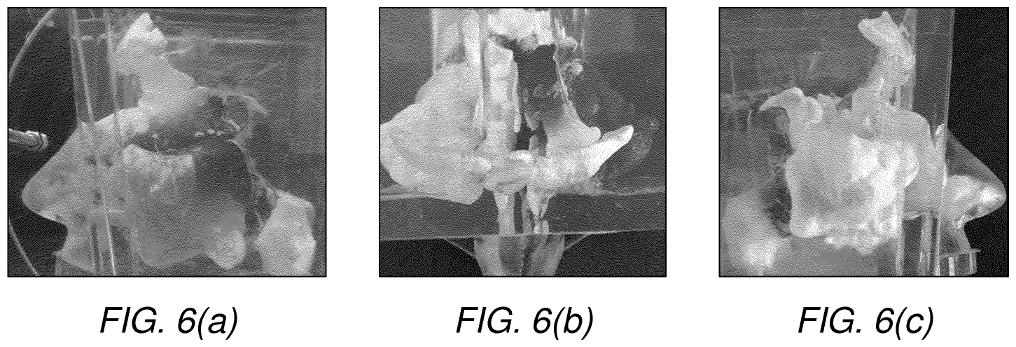

FIGS. 6(a) to (c) illustrate left lateral, superior and right lateral views, respectively, following delivery using an exhalation delivery system (EDS) device (XHANCETM, 0.1 mL×2) into the left nostril in a Draf 2 cast;

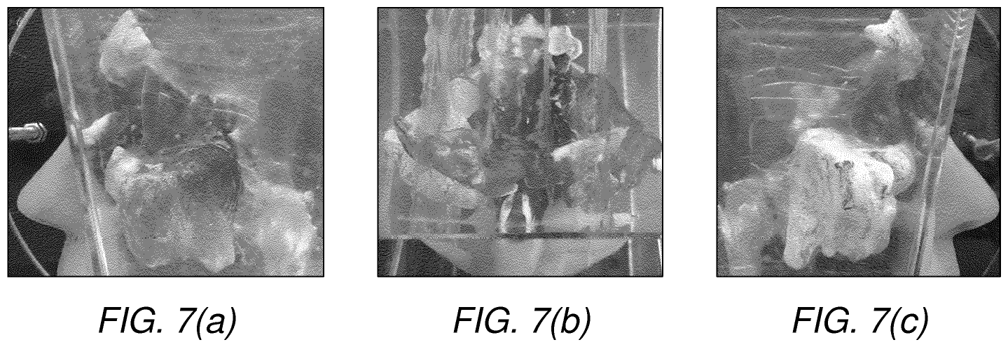

FIGS. 7(a) to (c) illustrate left lateral, superior and right lateral views, respectively, following delivery using an exhalation delivery system (EDS) device (XHANCE™, 0.1 mL×2) into the left nostril in a Draf 3 cast;

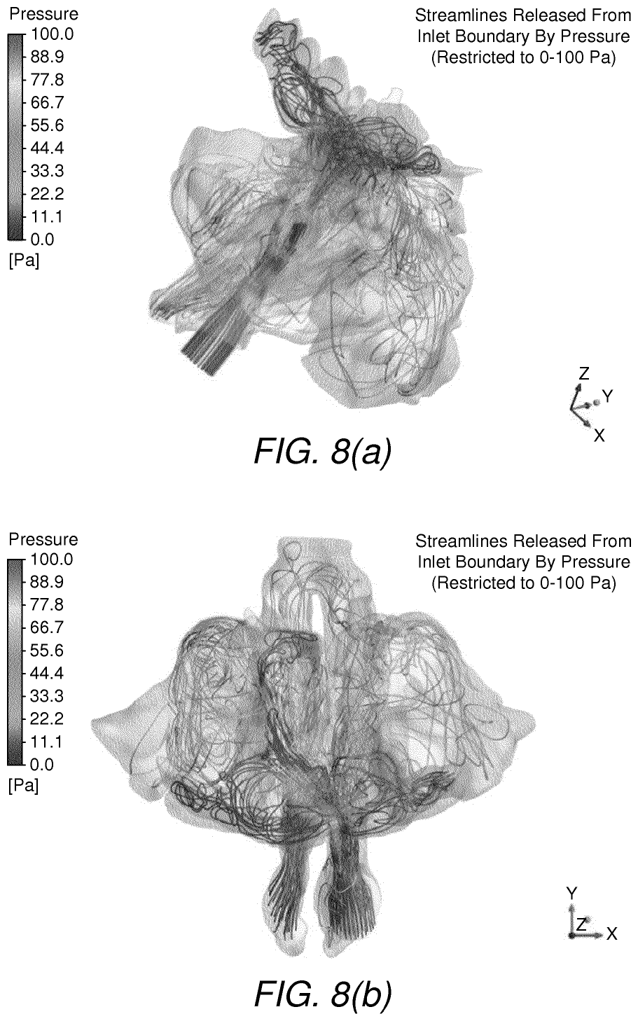

FIGS. 8(a) and (b) illustrate left lateral and superior models of the inlet streamlines colored by pressure at 40 Lmin⁻¹ using computational fluid dynamics (CFD) modelling in a Draf 3 cast; and

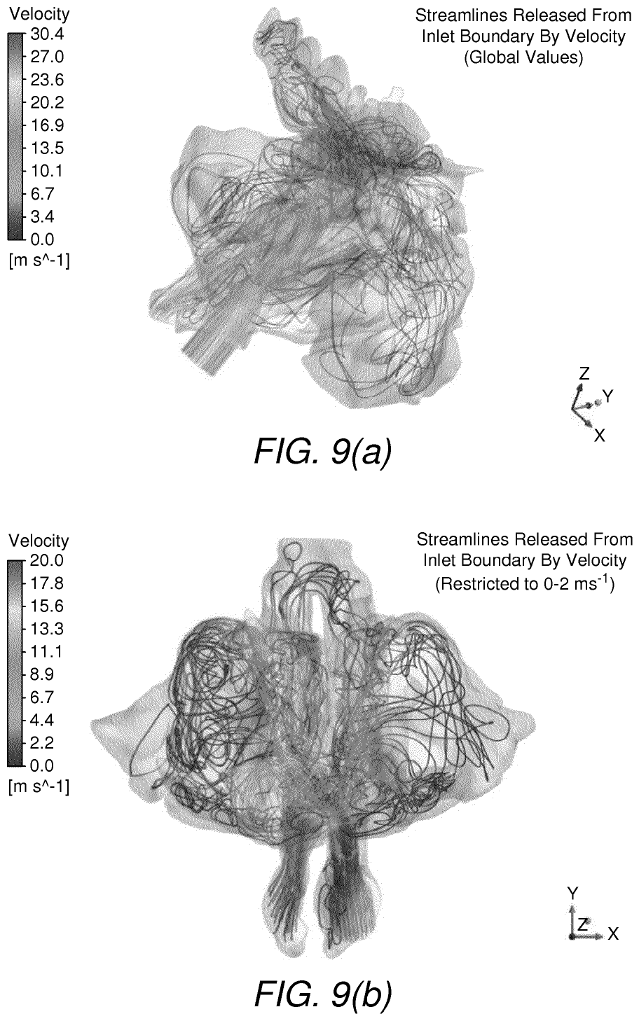

FIGS. 9(a) and (b) illustrate left lateral and superior models of the inlet streamlines colored by velocity at 40 Lmin⁻¹ using computational fluid dynamics (CFD) modelling in a Draf 3 cast.

The present invention will now be described by way of example only with reference to the following non-limiting Example.

### EXAMPLE

A study was performed to assess and compare deposition in anatomically-correct postsurgical Draf 2 and Draf 3 nasal casts following standard nasal spray delivery, EDS delivery and HVLF delivery in different head (vertex) positions (nasal floor horizontal=0°, 45° or 90°).

Two silicone casts of the nasal cavity, including sinuses, representing the geometry of a 47-year-old male patient with CRS who had undergone revision ESS of the maxillary, ethmoid and frontal sinuses, including first a Draf 2 procedure and a subsequent Draf 3 procedure, were made from a computed tomography (CT) scan using 3D printing. DICOMT^{M} files of the nasal cavities were loaded into medical imaging software, Slicer 4.6.2 (Harvard University, Cambridge, Mass.; open source). The software image editor and model maker were used to connect the DICOM files into a 3D geometry to build a model with 3D surfaces representing the nasal cavities. The 3D surface data, derived from hundreds of thousands of small triangles, was exported from Slicer as an STL file. The STL file was imported into the 3D tool software Meshmixerm (Autodesk, San Rafael, Calif.), which was used to make a coherent 3D representation of the nasal cavities in preparation for 3D printing. It was necessary to regularize and, in some areas, stitch together the triangles that make the 3D surface, remove artefacts, and remove surfaces that were not related to the nasal cavities. The analysis module in Meshmixer™ was used in order to ensure that the surface was complete and that the STL file could be created into an STL solid surface file. The STL solid surface file was exported and sent to 3D printing. The nasal geometry was made with a stereolithographic 3D printer (Objet 250, as supplied by CATI, Buffalo Grove, Ill.). This geometry was placed in a box, which was subsequently filled with a fluid, semi-transparent, semi-soft silicone (Andersen, Jessheim, Norway). After the silicone had dried, the silicone block was sectioned and the rigid stereolithographic material representing the nasal geometry was carefully broken into pieces and removed, leaving a transparent silicone replica of the postsurgical sinonasal cavity.

Experiments were first performed with the Draf 2 cast. HVLF delivery and standard nasal spray delivery were performed with the nasopharynx patent. EDS delivery was performed with a plug inserted in the nasopharynx in order to simulate velum closure, which occurs naturally during EDS delivery. Velum closure is desirable during HVLF sinonasal irrigations, but is not always achieved in real-life conditions—as verified by liquid escaping into the oral cavity.

A special jig was used which allowed fixation of the cast in standard positions (nasal floor at 0°, 45° and 90° relative to the horizontal plane). Cast filling and changes in surface color were captured and documented photographically.

The internal surfaces of the casts were coated with a sensitive gel that changes color on contact with liquid (Sar-Gel™, as supplied by Sartomer, Exton, Pa.). For the standard nasal spray, two sprays (0.1 mL each) of a commonly-available commercial steroid nasal spray (Nasonex™, as supplied by Merck, N.J.) were administered through one nostril to one side of the cast. For the EDS administration, two sprays (0.1 mL each) of fluticasone from an EDS device (XHANCE™, as supplied by OptiNose US, PA) were administered through one nostril to a single side of the cast with a tube connected to the mouthpiece so the investigator could exhale into the device during administration. For HVLF delivery, approximately 80 mL was administered using a squeeze bottle through one nostril to one side of the cast with inclination of the nasal floor first at 45° and then at 90°. A steady-state liquid level with liquid escaping from the contralateral nostril and/or the nasopharynx was reached in all HVLF experiments.

Previous validation work with nasal sprays and Sar-Gel™ had shown that the rate of color change is influenced by the time from exposure and volume of liquid reaching the surface¹⁷; therefore, spray and EDS delivery were evaluated at a standardized 10 minutes after administration.

As expected, the standard intranasal corticosteroid spray deposited liquid only in the anterior nasal segments, with similar deposition profiles in both the Draf 2 and Draf 3 casts, as illustrated in FIGS. 2 and 3, respectively.

HVLF irrigation exhibited different deposition profiles in the Draf 2 and Draf 3 casts, and the head (vertex) position had a significant effect on which sinuses were exposed to the irrigation liquid, as illustrated in FIGS. 4 and 5, respectively. As illustrated in FIGS. 4(a) and (b), distribution of HVLF irrigation liquid in the Draf 2 cast was characterized by penetration of the maxillary sinuses, but not the frontal sinuses or ethmoid region, at a 45° head position. When the head was tilted at a full 90°, distribution in the Draf 2 cast increased to include most of the frontal sinus and maxillary sinus, but still did not include the posterior wall of the ethmoid region. As illustrated in FIGS. 5(a) and (b), HVLF delivery in the Draf 3 cast at a 45° head position was characterized by maxillary sinus penetration, but no frontal sinus or ethmoid region penetration; and, when positioned at a 90° tilt, irrigation liquid was deposited in the frontal sinus, but there was no distribution to the maxillary sinus or ethmoid region due to the liquid spilling over to the contralateral side of the nose through the region where the nasal septum had been resected.

As illustrated in FIGS. 6 and 7, distribution with the EDS device was generally similar in the Draf 2 and Draf 3 casts: liquid deposition being observed throughout the nasal cavity and in the surgically-opened ethmoid and maxillary spaces. Regarding the frontal sinuses, there was limited penetration to the frontal sinus with EDS liquid delivery in the Draf 2 cast, as illustrated in FIGS. 6(a) and (b), whereas, in the Draf 3 cast, deposition was observed in the frontal recess and unified frontal sinuses, as illustrated in FIGS. 7(a) and (b).

This study demonstrates that both HVLF and EDS delivery produce substantially deeper intranasal deposition compared with standard nasal spray delivery. Furthermore, contrary to that expected, EDS delivery exhibited significant distinctions in sinus deposition as compared to HVLF irrigation. In the Draf 2 cast, deposition with HVLF irrigation is characterized by penetration into the maxillary sinuses, with increased penetration to the frontal sinuses when head tilt is increased from 45° to 90°. Deposition with the EDS device in the Draf 2 cast occurs in the maxillary space, but also in the ethmoid space, with limited delivery to the frontal sinuses. In Draf 3 post-surgical anatomy, HVLF delivery to the maxillary sinus spaces is actually reduced compared to Draf 2 anatomy due to the passage of liquid through the resected septum at the 90° head tilt. HVLF delivery to the frontal sinuses improves with a 90° head tilt, but access to the maxillary and ethmoid regions is reduced. Significantly, in the Draf 3 cast, EDS delivery is associated with deposition to the frontal sinuses along with the maxillary and ethmoid sinuses.

Although the conventional understanding of those skilled in the art was that more extensive surgery leads to increased sinus penetration with nasal irrigation, the findings of the present inventors suggest that the resected septum can act as an “escape path”, allowing premature exit of irrigated fluid, limiting the potential for mechanical lavage and drug delivery into some sinuses frequently affected by chronic inflammation in CRS. Unlike irrigation delivery, frontal sinus deposition resulting from EDS delivery does not appear to be negatively affected by the resected septum and may even benefit from the resection as a conduit to the frontal sinuses.

With the use of an EDS device in the Draf 3-operated patient, air flow carrying medication enters the opened ethmoid space, primarily turns laterally into the large open maxillary sinuses, and then flows up into the frontal recess and unified frontal sinuses via the large opening in the septum.

FIGS. 8 and 9 illustrate views of the inlet streamlines colored by pressure and velocity, respectively, at 40 Lmin⁻¹ using computational fluid dynamics (CFD) modelling in the Draf 3 cast, and show higher pressures and velocities to the frontal sinuses.

Although HVLF treatment offers a good method for sinonasal irrigation and lavage, it is clear from this study that HVLF delivery is an inefficient approach to drug delivery due to the small fraction (˜3%, ˜6-7 mL) of fluid retained in the nasal cavity following administration.⁸ This implies that, for example, with 1 mg of budesonide diluted in a single administration, one might expect 30 to 40 μg to remain in the nose/sinuses, which would be less than the delivered dose of budesonide nasal spray for allergic rhinitis (albeit with a presumably superior pattern of distribution). In contrast, no liquid was observed to escape from the nasal/sinus spaces following drug administration by the EDS device into the nasal casts, indicating that the full dose (372 μg [93 μg per actuation], XHANCE™) remains in the nasal passages and sinus cavities. Adjusting for the 1.7× higher glucocorticoid receptor binding affinityl⁸ of fluticasone propionate, 372 μg is a comparatively high dose in the target region.^{14,15,19}

EDS delivery of a comparatively-high concentration of a high-potency corticosteroid to sites of chronic inflammation in CRS that are not efficiently or consistently accessed with standard nasal delivery approaches can improve treatment outcomes in patients affected by inflammation even in the sinuses. A small, randomized, controlled study has reported significant clinical improvements from baseline in a previously-operated, but persistently symptomatic, patient population, with a trend towards magnetic resonance imaging-assessed reduction in paranasal sinus inflammation after twelve (12) weeks of treatment with EDS delivery of fluticasone versus placebo.¹³ Additionally, large, recently-published, controlled trials including CRS patients with nasal polyps with and without prior surgery, most of whom were symptomatic despite having previously tried intranasal steroid sprays, have demonstrated that EDS delivery of fluticasone improved a wide range of symptoms (including sense of smell), reduced polyp size, eliminated polyps in some patients (i.e. grade 0 in at least 1 nostril), and significantly-improved quality of life and functioning as measured by the 22-item Sino-Nasal Outcome Test, 36-item Short Form Health Survey, and other instruments in both patients with and without prior surgery.^{14,15,20,21}

Efficient and effective delivery of drug to the sinuses and to superior and posterior nasal regions is one important goal of sinus surgery. In Draf 2- and Draf 3-operated anatomy, both HVLF and EDS delivery produce improved deposition in the sinuses and the posterior/superior nasal cavity as compared to standard nasal spray. HVLF delivery is, however, impacted by head position and produced a different deposition pattern in Draf 2 and Draf 3 anatomy. EDS delivery produced generally similar deposition patterns in both Draf 2- and Draf 3-operated anatomy, with liquid deposition in the surgically-opened ethmoid and maxillary spaces. There was limited deposition in the frontal sinus spaces in the Draf 2 cast, but observable deposition in the Draf 3 cast with EDS delivery. The partial septum resection in Draf 3 surgery resulted in premature loss of liquid with HVLF delivery, which is consistent with findings in CFD simulations.

Finally, it will be understood that the present invention has been described in its preferred embodiments and can be modified in many different ways without departing from the scope of the invention as defined by the appended claims.

### REFERENCES

1. Barham H P, Ramakrishnan V R, Knisely A K, et al. Frontal sinus surgery and sinus distribution of nasal irrigation. Int Forum Allergy Rhinol. 2016; 6:238-242
2. Orlandi R R, Kingdom T T, Hwang P H, et al. International consensus statement on allergy and rhinology: rhinosinusitis. Int Forum Allergy Rhinol. 2016; 6 suppl 1:S22-209.
3. Abadie W M, McMains K C, Weitzel E K. Irrigation penetration of nasal delivery systems: a cadaver study. Int Forum Allergy Rhinol. 2011; 1:46-49.
4. Harvey R, Hannan S A, Badia L, Scadding G. Nasal saline irrigations for the symptoms of chronic rhinosinusitis. Cochrane Database Syst Rev. 2007; (3): CD006394.
5. Zhao K, Craig J R, Cohen N A, Adappa N D, Khalili S, Palmer J N. Sinus irrigations before and after surgery—visualization through computational fluid dynamics simulations. Laryngoscope. 2016; 126:E90-E96.
6. Kennedy D W. Additional options in chronic rhinosinusitis management. Int Forum Allergy Rhinol. 2018; July 20. doi: 10.1002/alr.22188.
7. Soudry E, Wang J, Vaezeafshar R, Katznelson L, Hwang P. Safety analysis of long term budesonide nasal irrigations in patients with chronic rhinosinusitis post endoscopic sinus surgery. Int Forum Allergy Rhinol. 2016; 6:568-572.
8. Harvey R J, Debnath N, Srubiski A, Bleiber B, Schlosser R J. Fluid residuals and drug exposure in nasal irrigation. Otolaryngol Head Neck Surg. 2009; 141:757-761.
9. Djupesland P G, Messina J, Mahmoud R. New exhalation delivery systems (EDS) enhance topical steroid delivery in chronic rhinosinusitis with nasal polyps (CSwNP). Poster session presented at: 2017 AAAAI Annual Meeting; Mar. 3-6, 2017; Atlanta, Ga.
10. Djupesland P G, Messina J, Mahmoud R. Enhanced nasal drug delivery with new exhalation delivery systems (EDS). Poster session presented at: 2016 ACAAI Annual Meeting; Nov. 10-14, 2016; San Francisco, Calif.
11. Djupesland P G. Nasal drug delivery devices: characteristics and performance in a clinical perspective—a review. Drug Deliv Transl Res. 2013; 3:42-62.
12. Djupesland P G, Zhao K, Messina J C, Mahmoud R A, Palmer J N. Exhalation delivery system provides superior deposition of liquid in post-surgical cavities in comparison to conventional spray or irrigation modalities. Poster session presented: 2018 ARS at COSM Meeting; Apr. 19-20, 2018; National Harbor, Md.
13. Hansen F S, Djupesland P G, Fokkens W J. Preliminary efficacy of fluticasone delivered by a novel device in recalcitrant chronic rhinosinusitis. Rhinology. 2010; 48:292-299.
14. Leopold D A, Elkayam D, Messina J C, Kosik-Gonzalez C, Djupesland P G, Mahmoud R A. NAVIGATE II: randomized, double-blind trial of the exhalation delivery system with fluticasone (EDS-FLU) for nasal polyposis. J Allergy Clin Immunol. 2018 Jun 19. pii: S0091-6749(18)30863-7. doi: 10.1016/j.jaci.2018.06.010.
15. Palmer J N, Jacobson K W, Messina J C, Kosik-Gonzalex C, Djupesland P, Mahmoud R A. EXHANCE-12: 1-year study of the exhalation delivery system with fluticasone (EDS-FLU) in chronic rhinosinusitis. Int Forum Allergy Rhinol. 2018; 8:869-876.
16. Han J K, Messina J C, Carothers J L, Djupesland P G, Mahmoud R A. EDS-FLU (Exhalation Delivery System With Fluticasone) Is Effective For Nasal Polyposis (NP) Patients With or Without History of Prior Surgery: Integrated Results from NAVIGATE I and II. Poster session presented: 2018 ARS at COSM Meeting; Apr. 19-20, 2018; National Harbor, Md.
17. Kundoor V, Dalby R N. Assessment of nasal spray deposition pattern in a silicone human nose model using a color-based method. Pharm Res. 2009; 27:30-36.
18. Daley-Yates P T. Inhaled corticosteroids: potency, dose equivalence and therapeutic index. Br J Clin Pharmacol. 2015; 80:372-380.
19. Djupesland P G, Vlckova I, Hewson G. Impact of baseline nasal polyp size and previous surgery on efficacy of fluticasone delivered with a novel device: a subgroup analysis. Am J Rhinol Allergy. 2010; 24:291-b 295.
20. Soteres D F Messina J, Carothers J, Mahmoud R, Djupesland P G. NAVIGATE I: a randomized double-blind trial of a fluticasone propionate exhalation delivery system (FLU-EDS) for Treatment of chronic rhinosinusitis with nasal polyps (CRSwNP). J Allergy Clin Immunol. 2017; 139 (2):AB66.
21. Sher M R, Mair E A, Messina J, Carothers J, Mahmoud R, Djupesland P G. EXHANCE-3: a phase 3, three-month study of safety and efficacy of fluticasone propionate exhalation delivery system (FLU-EDS) in patients with chronic rhinosinusitis with (CRSwNP) and without nasal polyps (CRSsNP). J Allergy Clin Immunol. 2017; 139 (2 suppl):AB66.

1. A method of treating a patient having a sinus condition, comprising, administering a therapeutic agent including a corticosteroid to the patient after the patient undergoes Draf 2 of Draf 3 surgery, wherein the therapeutic agent is administered via an exhalation delivery device that comprises a mouthpiece and a nosepiece, the nosepiece fitted to a first nostril of the patient, wherein the patient exhales into the mouthpiece to create a fluid flow out of the nosepiece and into the first nostril while the therapeutic agent is administered to the patient.

2. The method of claim 1, wherein the corticosteroid is fluticasone.

3. The method of claim 1, wherein the corticosteroid is fluticasone propionate.

4. The method of claim 1, wherein the surgery is Draf 2 surgery.

5. The method of claim 1, wherein the surgery is Draf 3 surgery.

6. The method of claim 1, wherein the therapeutic agent is in the form of a liquid aerosol.

7. The method of claim 6, wherein the therapeutic agent is administered using a spray pump.

8. The method of claim 1, wherein the therapeutic agent is in the form of a powder.

9. The method of claim 1, wherein the therapeutic agent is delivered in an amount of up to 400 μg.

10. The method of claim 1, wherein the therapeutic agent is delivered in an amount of up to 200 μg.

11. The method of claim 1, wherein the therapeutic agent is delivered in an amount of up to 100 μg.

12. The method of claim 1, wherein the therapeutic agent is delivered in an amount of at least 50 μg.

13. The method of claim 1, further comprising controlling a duration, rate or pressure of the fluid flow.

14. The method of claim 13, comprising controlling the fluid flow duration to be in a range of from about 2 seconds to about 3 seconds.

15. The method of claim 13, comprising controlling the fluid flow rate to be at least 10 Lmin⁻¹.

16. The method of claim 1, wherein the nosepiece is fitted to a second nostril of the patient, wherein the patient exhales into the mouthpiece to create a fluid flow out of the nosepiece and into the second nostril while the therapeutic agent is administered to the patient.

17. The method of claim 16, wherein the first and second nostrils are different nostrils of the patient or the same nostril of the patient.

18. (canceled)

19. The method of claim 18, wherein the surgery is endoscopic sinus surgery.

20. The method of claim 1, wherein the sinus condition is chronic rhinosinusitis (CRS).

21. The method of claim 9, wherein the therapeutic agent is delivered twice daily.

22. The method of claim 10, wherein the therapeutic agent is delivered twice daily.

23. The method of claim 11, wherein the therapeutic agent is delivered twice daily.

24. The method of claim 12, wherein the therapeutic agent is delivered twice daily.
